(19) United States
(12) Patent Application Publication
(10) Pub. No.: US 2007/0268999 A1
(43) Pub. Date: Nov. 22, 2007

(54) Apparatus and method for creating tomosynthesis and projection images

(75) Inventors: Christer Ullberg, Sollentuna (SE); Tom Francke, Sollentuna (SE)

Correspondence Address: HARNESS, DICKEY & PIERCE, P.L.C., P.O. BOX 8910, RESTON, VA, 20195, US

(73) Assignee: XCounter AB

(21) Appl. No.: 11/447,901

(22) Filed: Jun. 7, 2006

(30) Foreign Application Priority Data
May 22, 2006 (SE) 0601135-7

(51) Int. Cl. H05G 1/60; A61B 6/00; G01N 23/00; G21K 1/12

(52) U.S. Cl. 378/21

(57) ABSTRACT

An apparatus for creating tomosynthesis and projection images of an object from tomosynthesis image data obtained in a single measurement comprising an X-ray apparatus provided for obtaining the tomosynthesis image data of the object in a single measurement, a device provided for creating a three-dimensional tomosynthesis image of the object from the tomosynthesis image data, and a device provided for creating a two-dimensional projection image of the object from the three-dimensional tomosynthesis image by means of projecting the three-dimensional tomosynthesis image on a plane.

### PRIORITY STATEMENT

This application claims priority under 35 U.S.C. § 119 to Swedish Patent Application No. 0601135-7, filed on May 22, 2006, in the Swedish Patent and Registration Office, the entire contents of which are incorporated herein by reference.

### FIELD OF THE INVENTION

The invention relates generally to an apparatus and a method for creating tomosynthesis and projection images of an object.

### BACKGROUND OF THE INVENTION AND RELATED ART

An X-ray medical diagnostic method such as mammography or general body imaging is a low-dose procedure that creates one or more images of a part of a patient such as a breast or any other organ thereof, which is to be examined, e.g. for detection of early stages of cancer.

The mammography diagnostic procedure generally includes obtaining two projection images of each of the patient's breasts, one from above and one from the side. A physician or radiologist then reviews the images of the breast, i.e., mammograms, to identify any breast cancer.

There is, however, a trend today towards different kinds of three-dimensional imaging of the object in order to provide more information of the object that is being examined. Tomosynthesis imaging, in which image data is acquired at different angles, is one of these three-dimensional imaging techniques. By e.g. shifting and adding the images, it is possible to reconstruct planes in the object being imaged that is parallel to each other to thereby form a three-dimensional image of the object.

One example of a detector apparatus for creating such image data is described in U.S. Pat. No. 6,940,942. The detector apparatus comprises a radiation source emitting radiation centered around an axis of symmetry; a radiation detector comprising a stack of line detectors, each being directed towards the divergent radiation source to allow a ray bundle of the radiation that propagates in a respective one of a plurality of different angles to enter the line detector; an object area arranged in the radiation path between the divergent radiation source and the radiation detector for housing the object; and a device for moving the radiation source and the radiation detector relative the object essentially linearly in a direction essentially orthogonal to the axis of symmetry, while each of the stack of line detectors is adapted to record a plurality of line images of radiation as transmitted through the object in a respective one of the plurality of different angles.

However, sometimes the physician or radiologist needs to compare the three-dimensional tomosynthesis image with a more familiar two-dimensional projection image of the object. In other circumstances the physician or radiologist may want to study the more familiar two-dimensional projection image only, e.g. to save time in the examination of the images.

### SUMMARY OF THE INVENTION

A main object of the invention is therefore to provide an apparatus and a method, respectively, for creating tomosynthesis and projection images of an object from tomosynthesis image data obtained in a single measurement.

In this respect there is a particular object to provide such an apparatus and such a method, which are uncomplicated and can produce high-quality three-dimensional tomosynthesis images and high-quality two-dimensional images such as two-dimensional projection images with high spatial resolution, signal-to-noise ratio, dynamic range, and image contrast, while the imaging object is exposed to a minimum radiation dose.

A yet further object of the invention is to provide such an apparatus and such a method, which are reliable, accurate, and inexpensive.

These objects, among others, are attained by apparatuses and methods as claimed in the appended claims.

An apparatus for creating tomosynthesis and projection images of an object from tomosynthesis image data obtained in a single measurement comprises an X-ray apparatus provided for obtaining the tomosynthesis image data of the object in a single measurement, a device provided for creating a three-dimensional tomosynthesis image of the object from the tomosynthesis image data, and a device provided for creating a two-dimensional projection image of the object from the three-dimensional tomosynthesis image by means of projecting the three-dimensional tomosynthesis image on a plane.

The device provided for creating a two-dimensional projection image is preferably arranged for projecting the three-dimensional tomosynthesis image on a plane by means of summing, for each of the pixels of the two-dimensional projection image, pixel values of pixels along a respective straight line in the three-dimensional tomosynthesis image, wherein the straight lines converge in a single point.

Yet preferably, the X-ray apparatus comprises an X-ray source that emits radiation photons, and the single point is located at a distance from the three-dimensional tomosynthesis image, which is identical to the distance that the X-ray source is located from the object during the single measurement.

Further, the apparatus may comprise a device provided for displaying the three-dimensional tomosynthesis image and the two-dimensional projection image.

The present invention thus provides for the creation of three-dimensional tomosynthesis and two-dimensional projection images of an object from tomosynthesis image data obtained in a single measurement. The two-dimensional projection image is formed from the three-dimensional tomosynthesis image and is of high quality. A physician or radiologist may study a single one of the two images and if needed he/she may compare the studied image with the other one of the images without having to call back the patient (in medical applications) or to perform another measurement.

According to a further aspect of the invention three-dimensional tomosynthesis and two-dimensional attenuation images of the object are created, wherein the tomosynthesis image data of the object is collected in a single measurement, a three-dimensional tomosynthesis image of the object is created from the tomosynthesis image data, wherein the three-dimensional tomosynthesis image comprises a plurality of stacked two-dimensional images, and a two-dimensional attenuation image of the object is created from the three-dimensional tomosynthesis image by means of selecting a single one, or adding multiple ones, but maybe not all, of the plurality of stacked two-dimensional images.

It shall be understood that during the collection of the tomosynthesis image data of the object image data may be collected that is sufficient to directly form a two-dimensional attenuation image where each of the image pixels are formed from a single detector element measurement. However, in order to not expose the object to high radiation doses the measurement is performed at low radiation flux and/or with short detection times, which generally means that such formed two-dimensional attenuation image would be too noisy to be used.

The present invention is applicable to all kind of X-ray medical applications including mammography and general body examinations. Further, the invention may find use in other technical fields such as material testing and baggage checking.

Further characteristics of the invention and advantages thereof, will be evident from the detailed description of preferred embodiments of the present invention given hereinafter and the accompanying FIGS. 1-4, which are given by way of illustration only and thus, are not limitative of the present invention.

### BRIEF DESCRIPTION OF THE DRAWINGS

FIG. 1 illustrates schematically, in a block diagram, an apparatus for creating tomosynthesis and projection images of an object according to an embodiment of the present invention.

FIG. 2 illustrates how the creation of a two-dimensional projection image of an object is performed by the apparatus of FIG. 1.

FIG. 3 illustrates schematically, in a top view, an example of an X-ray apparatus for use in the apparatus of FIG. 1.

FIGS. 4a-c illustrate each schematically, in a top view, a particular X-ray bundle as it traverses the object during scanning by the X-ray apparatus of FIG. 3.

### DESCRIPTION OF PREFERRED EMBODIMENTS

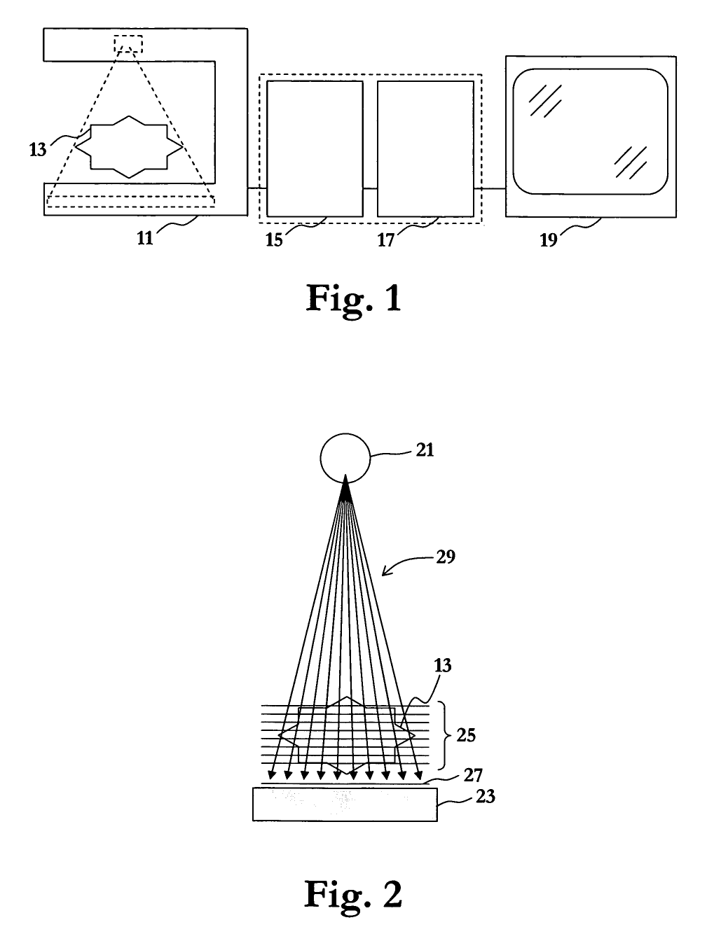

The apparatus of FIG. 1 comprises an X-ray apparatus 11 for obtaining tomosynthesis image data of an object 13, a reconstruction device 15 for creating a three-dimensional tomosynthesis image of the object 13 from the tomosynthesis image data, a projection image construction device 17 for creating a two-dimensional projection image of the object 13 from the three-dimensional tomosynthesis image, and a display device 19 for displaying the three-dimensional tomosynthesis image and the two-dimensional projection image.

The X-ray apparatus comprises generally an X-ray source 21 and an X-ray detector 23 as being illustrated in FIG. 2 and is provided for obtaining the tomosynthesis image data in a single measurement, in which image data is acquired at different angles. Details of how the measurement may be performed will be given further below in this description.

The reconstruction device 15 for creating a three-dimensional tomosynthesis image of the object 13 from the tomosynthesis image data may e.g. be any device known in the art. The reconstruction may be based on e.g. shift-and-add, filtered back projection, Fourier, or iterative methods for calculating the attenuation in the object 13 in three dimensions. Typically, the three-dimensional tomosynthesis image is obtained in the shape as a stack of parallel two-dimensional images 25 as being illustrated in FIG. 2. The tomosynthesis image could also be in the shape of a three dimensional model of the object segmented into three dimensional sub volumes, so called “voxels”. The voxels are preferably, but necessarily placed in parallel layers parallel to the lines 25.

The projection image construction device 17 is arranged for creating the two-dimensional projection image of the object 13 by means of projecting the three-dimensional tomosynthesis image on a first plane. Hereby, a high-quality two-dimensional projection image with high spatial resolution, signal-to-noise ratio, dynamic range, and image contrast is obtained.

Preferably, the two-dimensional projection image is formed by means of summing, for each of the pixels of the two-dimensional projection image 27, pixel values of pixels along a respective straight line 29 in the three-dimensional tomosynthesis image 25 as shown in FIG. 2. The converge in a single point, which is located at a distance from the three-dimensional tomosynthesis image 25, which is identical to the distance that the X-ray source 21 is located from the object 13 during the single measurement. This is schematically indicated in FIG. 2.

The geometry that is reconstructed is cone-shaped with an almost point-shaped X-ray source 21 at the top, from which a cone-shaped X-ray bundle of radiation is originating. The X-ray bundle traverses the object 13 and strikes the X-ray detector 23. The straight lines 29 thus coincide with the propagation path of radiation photons of the cone-shaped X-ray bundle.

If the tomosynthesis image is in the form of a reconstructed three dimensional model of the object, the summation could alternatively be done in any direction through the three dimensional model. For instance, the summation could be done in along parallel lines direction through the three dimensional model, e.g. perpendicular to the planes 25.

Further, the invention does not exclude that the two dimensional projection image is calculated in the same way as a three dimensional model of the object is calculated, but where the three dimensional model only consists of a single layer of voxels. The attenuation of X-rays in each voxel is then a representation of the two dimensional projection image.

Thus, according to a further embodiment of the invention three-dimensional tomosynthesis and two-dimensional attenuation images of the object 13 are created, wherein the tomosynthesis image data of the object is collected in a single measurement, a three-dimensional tomosynthesis image 25 of the object is created from the tomosynthesis image data, wherein the three-dimensional tomosynthesis image 25 comprises a plurality of stacked two-dimensional images (in some sense all three-dimensional images can be seen as a stack of two-dimensional images), and a two-dimensional attenuation image of the object is created from the three-dimensional tomosynthesis image by means of selecting a single one of the plurality of stacked two-dimensional images.

Alternatively, multiple ones, but maybe not all, of the plurality of stacked two-dimensional images are added to create the two-dimensional attenuation image of the object 13.

The reconstruction device 15 and the projection image construction device 17 may be integrated as software program modules in a common apparatus such as a microcomputer. The microcomputer and/or the display device 19 may further be integrated into the X-ray apparatus 11 or may be separate devices.

The microcomputer is advantageously provided for displaying the three-dimensional tomosynthesis image and the two-dimensional projection image simultaneously side by side on the display device 19 so that the physician or radiologist will be able to compare the three-dimensional tomosynthesis image with the more familiar two-dimensional projection image.

More advantageously though, the microcomputer comprises input means, e.g. a keyboard, a pointing device, or voice command receiving means for receiving user selections, and the display device 19 is provided for displaying the two-dimensional projection image in response to a first user selection through the input means and for displaying the three-dimensional tomosynthesis image in response to a second subsequent user selection through the input means. Hereby, the physician or radiologist is able to study the more familiar two-dimensional projection image firstly, and then if something suspicious is found, the physician or radiologist may select to display the three-dimensional tomosynthesis image without having to perform a second measurement. The microcomputer may have means for displaying the three-dimensional tomosynthesis image in various manners and layouts and means for displaying several three-dimensional tomosynthesis images from different angles—one after the other or several at the same time.

Further, the projection image construction device 17 may be arranged for creating a second two-dimensional projection image of the object 13 from the three-dimensional tomosynthesis image 25 by means of projecting the three-dimensional tomosynthesis image on a second plane, wherein the first and second planes are non-parallel. Hereby, a second two-dimensional projection image of the object 13 at another view angle is obtained. Such second two-dimensional projection image may be of importance to the physician or radiologist, not at least in mammography applications.

If the X-ray apparatus 11 is provided for obtaining the tomosynthesis image data at angles defining an angular range of at least 90°, the first and second planes may be substantially perpendicular to each another. In case of mammography two two-dimensional projection images may be taken of each of the patient's breast—one two-dimensional projection image from above and one two-dimensional projection image from the side.

It shall be appreciated that in case the three-dimensional tomosynthesis image is formed by a three-dimensional set of pixels or picture elements, each in the shape of e.g. a cube or cuboid, and each representing the X-ray attenuation in a corresponding voxel of the object 13, each of the summations of pixel values of pixels along a respective straight line in the three-dimensional tomosynthesis image may be weighted depending on how the straight line passes or cuts through the pixels. For example, if the straight line passes or cuts through a pixel in the middle thereof the pixel value of that pixel should be given high weight in the summation, whereas if the straight line passes or cuts through a corner portion of a pixel the pixel value of that pixel should be given low weight in the summation.

Generally, if the pixels in the three-dimensional tomosynthesis image are cubic or cuboidic, the weight of the pixel value of each cubic or cuboidic pixel along each straight line may depend on the length of the straight line that is within the cubic or cuboidic pixel.

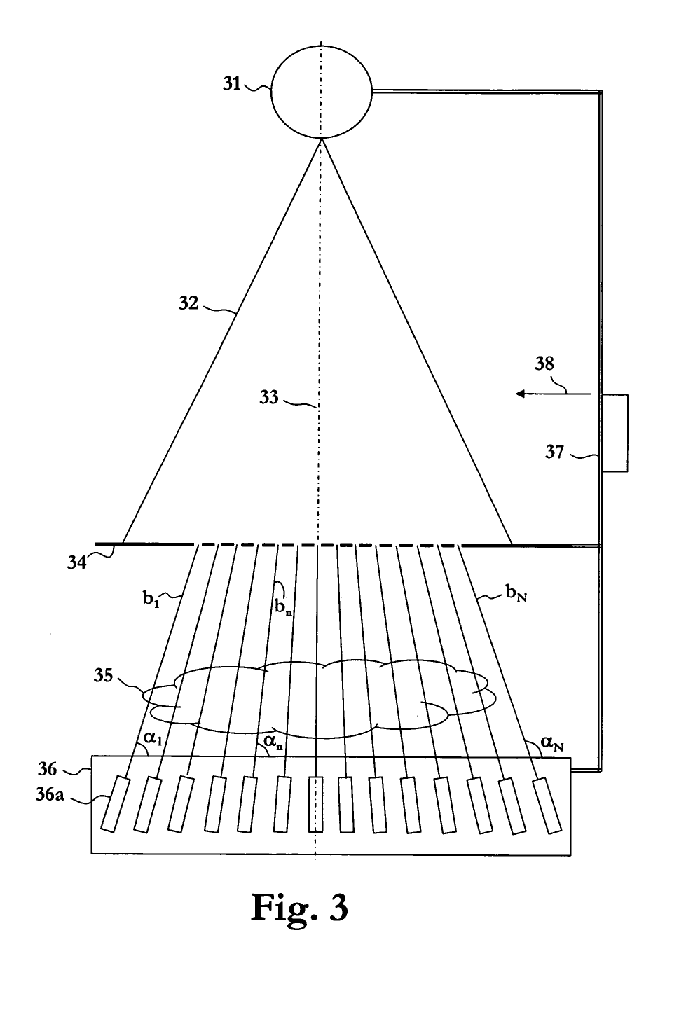

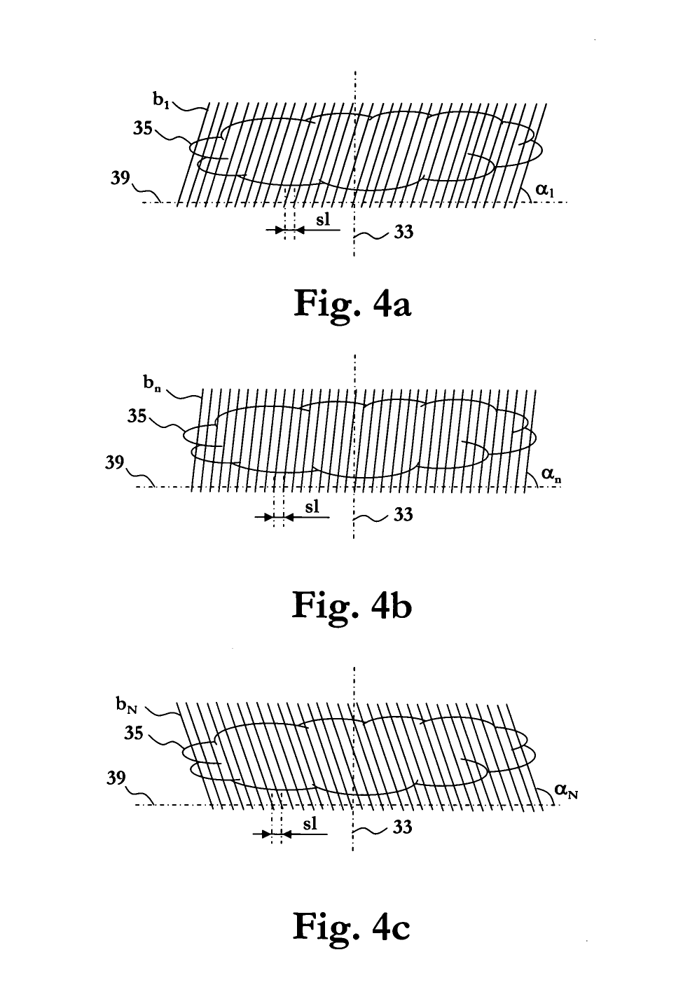

With reference now to FIGS. 3 and 4 an example of an X-ray apparatus for use in the apparatus of FIG. 1 will briefly be described.

The X-ray apparatus comprises a divergent X-ray source 31, which produces X-rays 32 centered around an axis of symmetry 33, a collimator 34, a radiation detector 36, and a device 37, which rigidly connects the X-ray source 31, the collimator 34, and the radiation detector 36 to each other and which moves the X-ray source 31, the collimator 34, and the radiation detector 36 linearly in direction 38 essentially orthogonal to the axis of symmetry 33 to scan scan an object 35, which is to be examined.

The radiation detector 36 comprises a stack of line detectors 36a, each being directed towards the divergent radiation source 31 to allow a respective ray bundle b₁, . . . , bₙ, . . . , b_{N} of the radiation 32 that propagates in a respective one of a plurality of different angles α₁, . . . , αₙ, . . . , α_{N} with respect to the front surface of the radiation detector 36 to enter the respective line detector 36a.

The collimator 34 may be a thin foil of e.g. tungsten with narrow radiation transparent slits etched away, the number of which corresponds to the number of line detectors 36a of the radiation detector 36. The slits are aligned with the line detectors 36a so that X-rays passing through the slits of the collimator 34 will reach the detector units 36a, i.e. as the respective ray bundles b₁, . . . , bₙ, . . . , b_{N}. The collimator 34, which is optional, prevents radiation, which is not directed directly towards the line detectors 36a, from impinging on the object 35, thereby reducing the radiation dose to the object 35. This is advantageous in all applications where the object 35 is a human or an animal, or parts thereof.

During scanning the device 37 moves the radiation source 31, the collimator 34, and the radiation detector 36 relative to the object 35 in a linear manner parallel with the front of the radiation detector as being indicated by arrow 38, while each of the line detectors 36a records a plurality of line images of radiation as transmitted through the object 35 in a respective one of the different angles α₁, . . . , αₙ, . . . , α_{N}.

The scanning may alternatively be performed by rotating the radiation source 31, the collimator 34, and the radiation detector 36 relative to the object 35. It shall also be appreciated that a similar scanning is obtained by holding the radiation source 31, the collimator 34, and the radiation detector 36 still and instead moving the object 35 to be examined.

The scanning of the object 35 is performed a length, which is sufficiently large so that each one of the line detectors 36a can be scanned across the entire object of interest to obtain, for each of the line detectors 6a, a two-dimensional image of radiation as transmitted through the object 35 in a respective one of the different angles α₁, . . . , αₙ, . . . , α_{N}.

In FIGS. 4a-c three different X-ray bundles b₁, bₙ, and b_{N} are schematically illustrated as they traverse the examination object 35 during scanning by the X-ray apparatus of FIG. 3. Reference numeral 39 indicates a plane parallel with the scanning direction 38 and with the front of the radiation detector 32.

As can be seen in FIGS. 4a-c each line detector/X-ray bundle pair produces a complete two-dimensional image at a distinct one of the different angles. FIG. 4a illustrates the formation of a two-dimensional image of radiation transmitted through the object at an angle α₁, FIG. 4b illustrates the formation of a two-dimensional image of radiation transmitted through the same object, but at an angle αₙ, and FIG. 4c illustrates the formation of a similar two-dimensional image, but at an angle α_{N}.

A preferred line detector for use in the X-ray apparatus of FIGS. 3 and 4 is a gaseous-based parallel plate detector, preferably provided with an electron avalanche amplifier. Such a gaseous-based parallel plate detector is an ionization detector, wherein electrons freed as a result of ionization by ionizing radiation are accelerated in a direction essentially perpendicular to the direction of the radiation.

For further details regarding such kind of gaseous-based line detectors for use in the present invention, reference is made to the following U.S. Patents by Tom Francke et al. and assigned to XCounter AB of Sweden, which patents are hereby incorporated by reference: U.S. Pat. Nos. 6,546,070; 6,522,722; 6,518,578; 6,118,125; 6,373,065; 6,337,482; 6,385,282; 6,414,317; 6,476,397; and 6,477,223.

It shall, nevertheless, be realized that any other line detector may be used in the X-ray apparatus of FIGS. 3 and 4. Such line detectors include scintillator-based arrays, CCD arrays, TFT- and CMOS-based detectors, liquid detectors, and solid-state detectors such as one-dimensional PIN-diode arrays with edge-on, near edge-on or perpendicular incidence of X-rays.

Still further, other X-ray apparatuses such as e.g. one including a two-dimensional flat panel detector for detection may be used in the apparatus of FIG. 1 and thus in the present invention. Such X-ray apparatus is rotated or tilted so that a number of two-dimensional projection images (e.g. 5-200) of the object are taken at different angles.

What is claimed is:

1. An apparatus for creating tomosynthesis and projection images of an object from tomosynthesis image data obtained in a single measurement comprising:
an X-ray apparatus provided for obtaining the tomosynthesis image data of the object in a single measurement;
a device provided for creating a three-dimensional tomosynthesis image of the object from the tomosynthesis image data; and
a device provided for creating a two-dimensional projection image of the object from the three-dimensional tomosynthesis image by means of projecting the three-dimensional tomosynthesis image on a first plane.

2. The apparatus of claim 1 wherein the device provided for creating a two-dimensional projection image is arranged for projecting the three-dimensional tomosynthesis image on a first plane by means of summing, for each of the pixels of the two-dimensional projection image, pixel values of pixels along a respective straight line in the three-dimensional tomosynthesis image, wherein the straight lines converge in a single point.

3. The apparatus of claim 2 wherein
the X-ray apparatus comprises an X-ray source that emits radiation photons; and
the single point is located at a distance from the three-dimensional tomosynthesis image, which is identical to the distance that the X-ray source is located from the object during the single measurement.

4. The apparatus of claim 1 wherein the apparatus comprises a device provided for displaying the three-dimensional tomosynthesis image and the two-dimensional projection image.

5. The apparatus of claim 4 wherein the device provided for displaying the three-dimensional tomosynthesis image and the two-dimensional projection image is arranged for displaying the three-dimensional tomosynthesis image and the two-dimensional projection image simultaneously side by side.

6. The apparatus of claim 4 wherein
said apparatus comprises input means; and
the device provided for displaying the three-dimensional tomosynthesis image and the two-dimensional projection image is arranged for displaying the two-dimensional projection image in response to a first user selection via the input means and for displaying the three-dimensional tomosynthesis image in response to a second, preferably subsequent, user selection via the input means.

7. The apparatus of claim 1 wherein the device provided for creating a two-dimensional projection image of the object is arranged for creating a second two-dimensional projection image of the object from the three-dimensional tomosynthesis image by means of projecting the three-dimensional tomosynthesis image on a second plane, wherein the first and second planes are non-parallel.

8. The apparatus of claim 7 wherein
the X-ray apparatus is provided for obtaining the tomosynthesis image data at angles defining an angular range of at least 90°, and
the first and second planes are substantially perpendicular to each another.

9. The apparatus of claim 1 wherein the X-ray apparatus comprises:
a divergent radiation source emitting radiation centered around an axis of symmetry;
a radiation detector comprising a stack of line detectors, each being directed towards the divergent radiation source to allow a ray bundle of the radiation that propagates in a respective one of a plurality of different angles to enter the line detector;
an object area arranged in the radiation path between the divergent radiation source and the radiation detector for housing the object; and
a device for moving the divergent radiation source and the radiation detector relative the object essentially linearly in a direction essentially orthogonal to the axis of symmetry, while each of the line detectors is adapted to record a plurality of line images of radiation as transmitted through the object in a respective one of the plurality of different angles, wherein
the device for moving is adapted to move the divergent radiation source and the radiation detector relative the object a length which is sufficient for scanning each of the line detectors across the entire object to obtain, for each of the line detectors, a two-dimensional image of radiation as transmitted through the object in a respective one of the plurality of different angles.

10. The apparatus of claim 9 wherein
the divergent radiation source is an X-ray source; and
the line detectors are each a gaseous-based ionization detector, wherein electrons freed as a result of ionization by a respective ray bundle are accelerated in a direction essentially perpendicular to the direction of that ray bundle.

11. A method for creating tomosynthesis and projection images of an object from tomosynthesis image data obtained in a single measurement comprising the steps of:
collecting the tomosynthesis image data of the object in a single measurement;
creating a three-dimensional tomosynthesis image of the object from the tomosynthesis image data; and
creating a two-dimensional projection image of the object from the three-dimensional tomosynthesis image by means of projecting the three-dimensional tomosynthesis image on a plane.

12. The method of claim 11 wherein the three-dimensional tomosynthesis image is projected onto a plane by means of summing, for each of the pixels of the two-dimensional projection image, pixel values of pixels along a respective straight line in the three-dimensional tomosynthesis image, wherein the straight lines converge in a single point.

13. An apparatus for creating three-dimensional tomosynthesis and two-dimensional attenuation images of an object from tomosynthesis image data obtained in a single measurement comprising:
an X-ray apparatus provided for obtaining the tomosynthesis image data of the object in a single measurement;
a device provided for creating a three-dimensional tomosynthesis image of the object from the tomosynthesis image data, wherein said three-dimensional tomosynthesis image comprises a plurality of stacked two-dimensional images; and
a device provided for creating a two-dimensional attenuation image of the object from the three-dimensional tomosynthesis image by means of selecting a single one, or adding multiple ones, of said plurality of stacked two-dimensional images.

14. A method for creating three-dimensional tomosynthesis and two-dimensional attenuation images of an object from tomosynthesis image data obtained in a single measurement comprising the steps:
collecting the tomosynthesis image data of the object in a single measurement;
creating a three-dimensional tomosynthesis image of the object from the tomosynthesis image data, wherein said three-dimensional tomosynthesis image comprises a plurality of stacked two-dimensional images; and
creating a two-dimensional attenuation image of the object from the three-dimensional tomosynthesis image by means of selecting a single one, or adding multiple ones, of said plurality of stacked two-dimensional images.
